(19) United States
(12) Plant Patent Application Publication
(10) Pub. No.: US 2017/0290226 P1
(43) Pub. Date: Oct. 5, 2017

(54) Artichoke plant named 'SJ 857'

(50) ;

(71) Applicant: Steve Jordan, Lompoc, CA (US)

(72) Inventor: Steve Jordan, Lompoc, CA (US)

(21) Appl. No.: 15/530,949

(22) Filed: Mar. 27, 2017

Related U.S. Application Data
(60) Provisional application No. 62/390,546, filed on Apr. 1, 2016.

(51) Int. Cl. A01H 5/12

(52) U.S. Cl. CPC A01H 5/02
USPC PLT/258

(57) ABSTRACT

A new cultivar of artichoke named ‘SJ 857’ that is the most vigorous and productive yielding variety of all French Lyon type Cynara varieties known to the inventor.

### CROSS-REFERENCE TO RELATED APPLICATIONS

This application claims priority of co-pending provisional application Ser. No. 62/390,546 as filed Apr. 1, 2016.

### STATEMENT REGARDING FEDERALLY SPONSORED RESEARCH OR DEVELOPMENT

None.

Genus and species: CYNARA scolymus.

Variety denomination: ‘SJ 857’.

### BACKGROUND AND SUMMARY OF THE VARIETY

The present invention relates to a new and distinct cultivar of artichoke plant known commonly as French Lyon artichoke, a perennial herb that is grown as a food crop for the production of edible vegetable delicacies. The new invention is known botanically as CYNARA scolymus and will be referred to hereinafter by the cultivar name ‘SJ 857’. Cynara is in the family Asteraceae, formerly known as Compositae.

An individual plant of Cynara scolymus ‘SJ 857’ is comprised of main stem, lateral stems, leaves, and immature flower buds known as involucra. Each involucrum is made up of imbricate phyllaries also known as involucral bracts. Freshly harvested immature flower buds can be baked, grilled, boiled, or steamed, making the fleshy part of the bracts, the fleshy receptacle, and the uppermost part of the peduncle, edible. If not harvested, but permitted to mature, flower buds will develop into flower heads known as capitula.

In 2011 a population of seeds were harvested from a cage of Lyon type Z-530 artichoke plants that were self-pollinated. The seedlings was planted in Lompoc, Calif., in an area where artichokes are commercially grown.

In April of 2012, a single superior plant was selected from that group of seedlings and the new Cynara variety was named ‘SJ 857.’ Additional plants of the ‘SJ 857’ variety were vegetatively asexually propagated (using the method of division) from the mother plant in 2012. In April of 2013, plant tissue of the ‘SJ 857’ variety was sent to the inventor's tissue culture lab, and in 2014 the ‘SJ 857’ variety was asexually propagated by tissue culture. Under careful observation, ‘SJ 857’ has been determined uniform, stable and true to type in subsequent generations of asexual propagation by both tissue culture and division.

### SUMMARY OF THE INVENTION

The following traits have been repeatedly observed and represent the distinguishing characteristics of the new Cynara cultivar named ‘SJ 857’. These traits in combination distinguish ‘SJ 857’ from all other existing varieties of French Lyon type Cynara known to the inventor. ‘SJ 857’ has not yet been tested under all possible conditions and phenotypic differences may be observed with variations in environmental, climatic, and cultural conditions, however, without any variance in genotype.

1. ‘SJ 857’ is the most vigorous and productive yielding variety of all French Lyon type Cynara varieties.

### BRIEF DESCRIPTION OF THE DRAWINGS

The accompanying color drawings illustrate the overall appearance of the new Cynara cultivar named ‘SJ 857’ showing color as true as is reasonably possible to obtain in color reproductions of this type. Color in the drawings may differ from color values cited in the detailed botanical description, which accurately describe the actual color of the new variety ‘SJ 857’. The drawings were made using conventional techniques and although immature flower bud and foliage color may appear different from actual color due to light reflectance, it is as accurate as possible by conventional photography.

FIG. 1 depicts a whole plant of ‘SJ 857’;

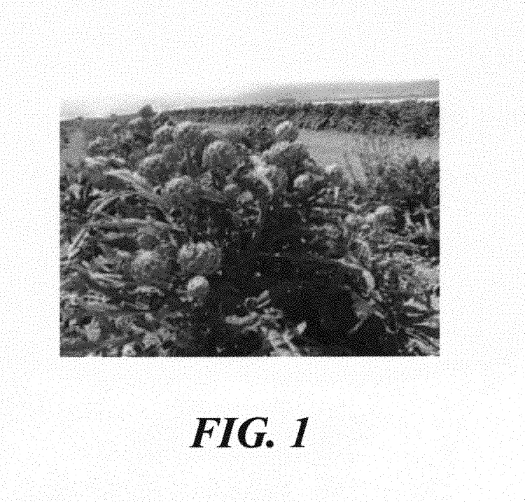

FIG. 2 depicts two mature heads of ‘SJ 857’ cut in half;

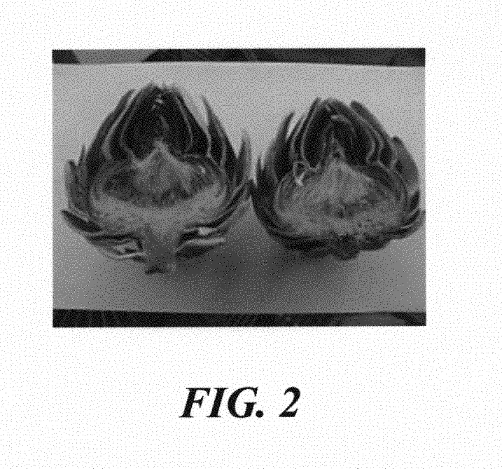

FIG. 3 depicts a mature flower of “SJ 857’;

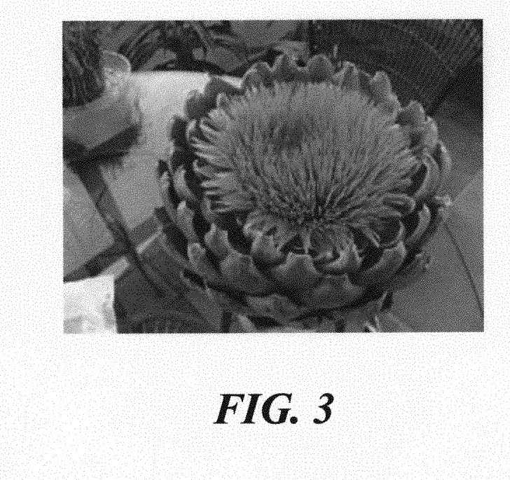

FIG. 4 depicts an immature flower bud of ‘SJ 857’; and

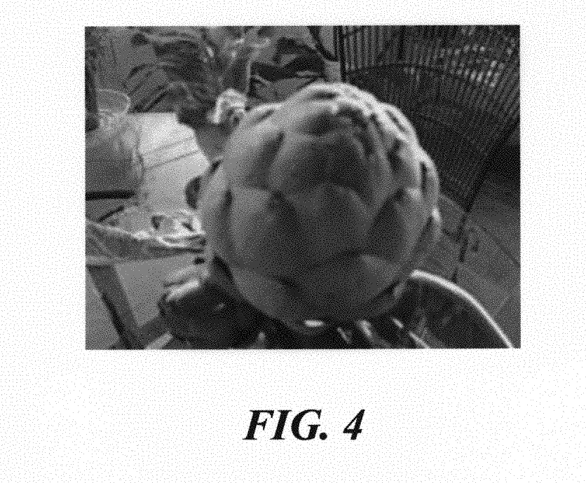

FIG. 5 depicts a row of ‘SJ 857’ plants.

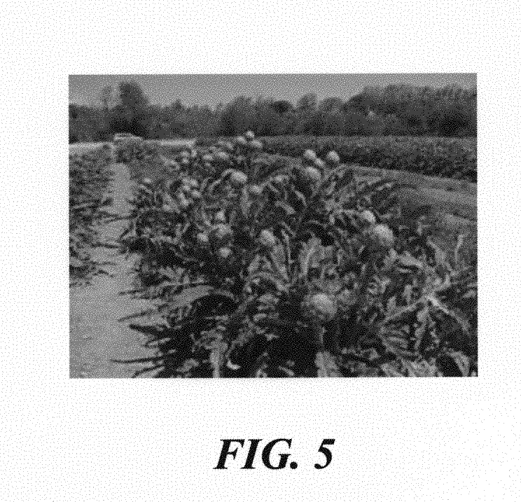

### DETAILED BOTANICAL DESCRIPTION

The following is a detailed botanical description of the new Cynara cultivar named ‘SJ 857’. Color determinations are in accordance with The 2001 Royal Horticultural Society Colour Chart of London, England, except where general color terms of ordinary dictionary significance are used. The following observations, measurements and values describe the new cultivar named ‘SJ 857’ as grown in Lompoc, Calif. under conditions used in horticultural practice.

Lompoc, Calif. is located on the central coast in Santa Barbara County. Conditions in Lompoc vary with air temperatures ranging from 15.5° Centigrade to 26.6° Centigrade. The relative humidity is generally high. Prevailing winds are northwesterly and rainfall averages twelve inches per year. Growing requirements are similar to the species.
Botanical classification: CYNARA scolymus ‘SJ 857’.
Family: Asteraceae (formerly Compositae).
Genus: CYNARA.
Species: scolymus.
Denomination: ‘SJ 857’.
Common name: French Lyon artichoke.
Commercial classification: Food crop.
Use: Production of edible vegetable delicacies.
Parentage: Cynara scolymus ‘SJ 857’ was a seedling selected in Lompoc, Calif., from a population of siblings grown from seed harvested from a self-pollinated plant of Cynara scolymus Z-530 variety of artichoke.

1. A new and distinct cultivar of artichoke plant named ‘SJ 857’ as described and illustrated herein.
